(19) United States
(12) Patent Application Publication
(10) Pub. No.: US 2015/0038840 A1
(43) Pub. Date: Feb. 5, 2015

(54) PORTABLE MEDICAL IMAGER WITH GUI INTERFACE COMPRISING A MOTION SENSOR

(71) Applicant: KONINKLIJKE PHILIPS N.V., EINDHOVEN (NL)

(72) Inventors: Rizwan Hassan, Danville, CA (US); Lingxiong Shao, Saratoga, CA (US); Steven Allen, Danville, CA (US)

(21) Appl. No.: 14/387,859

(22) PCT Filed: Mar. 20, 2013
(86) PCT No.: PCT/IB2013/052199

§ 371 Date: Sep. 25, 2014

Related U.S. Application Data
(60) Provisional application No. 61/617,696, filed on Mar. 30, 2012.

(51) Int. Cl. A61B 6/00

(52) U.S. Cl. CPC A61B 6/527; A61B 6/4258; A61B 6/4405; A61B 6/469; A61B 6/4028; A61B 6/466; A61B 6/4216; A61B 6/5205
USPC 600/436

(57) ABSTRACT

A portable imaging system for imaging a region of interest (ROI) includes a housing (12), a radiation detector(16)mounted to the housing (12), the detector generating radiation data indicating a location of gamma photon strikes on the detector, a motion sensor (20) which senses motion of the detector and outputs motion data indicative of the location and orientation of the detector at the time of each gamma photon strike, and at least one processor (22, 26) programmed to receive the radiation data from the radiation detectors (16) and the motion data from the motion sensor (20) and reconstruct a 3D volume image of the ROI from the received radiation and motion data.

The present application relates generally to nuclear medical imaging. It finds particular application in conjunction with single-photon emission computed tomography (SPECT) and positron emission tomography (PET) and will be described with particular reference thereto. However, it is to be understood that it also finds application in other usage scenarios such as x-ray, ultrasound, light, and magnetic resonance scenarios and is not necessarily limited to the aforementioned application.

SPECT is a nuclear medical imaging technique that employs a radioisotope injected into a patient to image a region of interest (ROI) of the patient. Typically, the radioisotope is combined with pharmaceutically-active molecules to create a radiopharceutical that is preferentially absorbed by specific types of tissue. The radioisotope undergoes gamma-ray decay at a predictable rate and characteristic energy. One or more radiation detectors are placed adjacent to the patient to monitor and receive emitted radiation.

To obtain a three-dimensional image, the radiation detectors are rotated or indexed around the patient to monitor the emitted radiation from a plurality of angles, thereby creating a plurality of two-dimensional images of radiation distributions at different angles. Traditionally, a gantry is employed to support and move the radiation detectors around the patient. Using the created two-dimensional images and the corresponding angles, a three-dimensional image is reconstructed. Challenges with employing traditional SPECT systems follow from the radiation detectors, which are bulky and heavy and require stable, expensive gantries that limit mobility. Gantries are utilized because the detectors are too heavy and are held over the patient for long periods of time in order to acquire an image. Further, most traditional SPECT system are supported by an articulating arm or mount on the ring gantry making it difficult for hospital staff to use a single detector on gurney stricken patients.

Currently, few types of portable nuclear medicine detectors are available which utilize either photomultiplier tubes or solid state detectors. These portable nuclear medicine detectors are typically used during surgery to detect if the surgeon has completely removed a tumor. The portable nuclear medicine detectors are typically connected to a computer that processes and displays the image. In addition, existing portable nuclear medicine detectors do not generate planar slice images. Existing portable medical imager devices use a gantry or an articulating arm. As solid state detector technology becomes more advanced, a need exists for a portable nuclear medicine imaging device which can utilize the above technologies and replace traditional nuclear systems and existing portable nuclear medicine detectors.

The present application provides a new and improved system which overcomes the above-referenced problems and others.

In accordance with one aspect, a portable imaging system for imaging a region of interest (ROI) is provided. The system including a housing, a radiation detector mounted to the housing, the detetor generating radiation data indicating a location of gamma photon strikes on the detector, a motion sensor which senses motion of the detector and outputs motion data indicative of the location and orientation of the detector at the time of each gamma photon strike, and at least one processor programmed to receive the radiation data from the radiation detectors and the motion data from the motion sensor and reconstruct a 3D volume image of the ROI from the received radiation and motion data.

In accordance with another aspect, a portable imaging system for imaging a region of interest (ROI) is provided. The system including a housing, a radiation detector mounted to the housing to generate radiation data indicating the location of gamma photon strikes, a display mounted to an opposite face of the housing, and a motion sensor mounted in or to the housing to sense movement of the housing and generate motion data indicative of the relative positions and orientations of the housing.

In accordance with another aspect, a method for imaging a region of interest (ROI) is provided. The method including positioning a portable imaging device facing a ROI of a patient, the portable imaging device including a detector disposed on the housing to generate radiation data indicating the location of gamma photon strikes and a motion sensor which senses motion of the detector and outputs motion data indicative of the location and orientation of the detector at the time of each gamma photon strike, receiving with at least one processor the radiation data from the radiation detectors and the motion data from the motion sensor, and reconstructing by the processor an image of the ROI from the received radiation and motion data.

One advantage resides in a portable medical imager used for planar, slice imaging purposes.

Another advantage resides a portable medical imager devices without a gantry or an articulating arm.

Another advantage resides in imaging, processing, reconstruction, and display of image data within a portable medical imager.

Still further advantages of the present invention will be appreciated to those of ordinary skill in the art upon reading and understand the following detailed description.

The invention may take form in various components and arrangements of components, and in various steps and arrangements of steps. The drawings are only for purposes of illustrating the preferred embodiments and are not to be construed as limiting the invention.

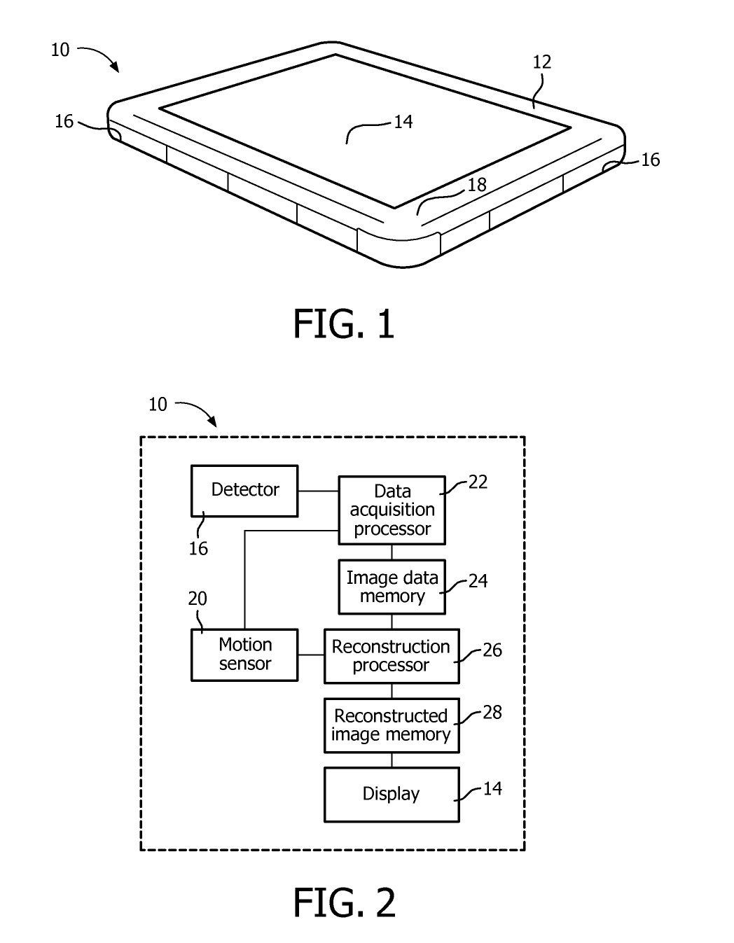

FIG. 1 illustrates a perspective diagram of a portable medical imaging system in accordance with present application.

FIG. 2 illustrates a portable medical imaging system in accordance with the present application.

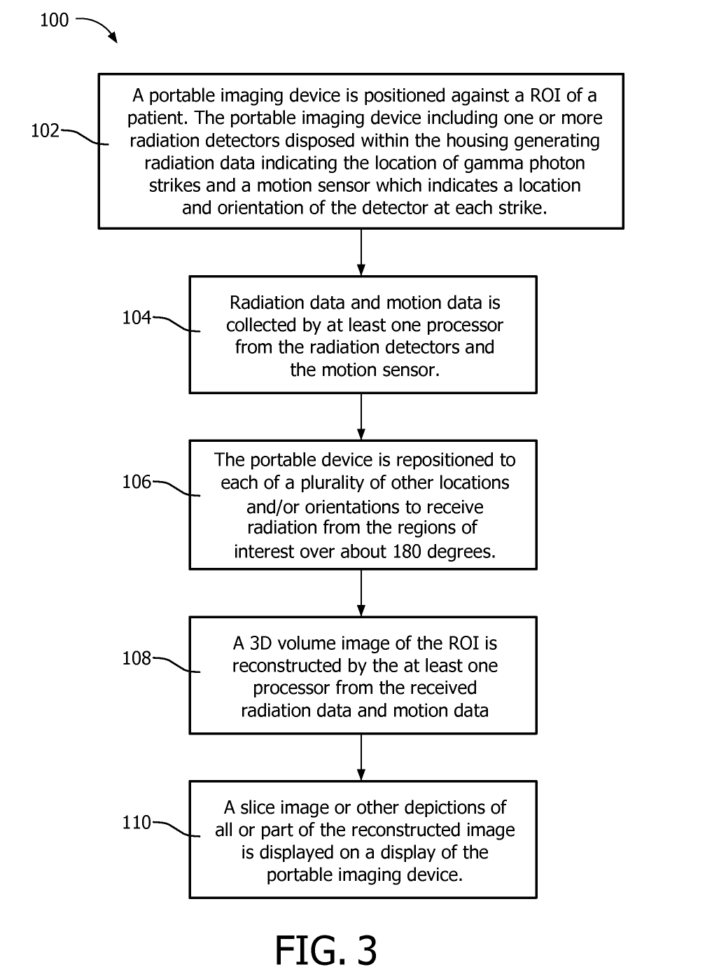

FIG. 3 illustrates a block diagram of a method of imaging a patient using a portable medical imaging system in accordance with the present application.

The present disclosure provides a portable single-photon emission computed tomography (SPECT) imaging system that does not need a stationary or rotating gantry. Instead, the system employs detector modules arranged in a frame of a portable medical imager 10. The system is particularly well suited for applications such as SPECT imaging the heart, brain, thyroid, bones, joints, ligaments, tendons, muscles, nerves, kidneys, lungs, and the like. During imaging, the portable medical imager 10 is positioned and held adjacent and facing a region of interest (ROI) of the patient's body for time in order to acquire a projection image. Projection images from a plurality of angles are reconstructed into a 3D volume image from which slice, surface rendering, and the like images can be generated.

With reference to FIGS. 1-2, a SPECT system includes a portable medical imager 10. The portable medical imager 10 is sized for portable operation and enables the user to hold the detector against the patient's body to acquire projection images. The portable medical imager 10 includes a notebook or tablet sized electronic device. With regards to being handheld, the portable medical imager 10 is operated solely by the user's hand(s), i.e., no gantry, supports, or articulating arms are needed. By being notebook or tablet sized, the device can be taken almost anywhere the user travels (e.g., the user is not limited by carrying a large, bulky and heavy device). In some embodiments, the portable medical imager 10 is extremely portable (e.g., small form factor, thin, low profile, lightweight). By way of example, the portable medical imager 10 may correspond to consumer electronic products such as hand held computers or tablet PCs.

In some embodiments, portable medical imager 10 performs a single function (e.g. acquires image data of a patient). In some embodiments, the portable medical imager 10 performs multiple functions (e.g. image acquisition and image processing.) In another embodiment, the portable medical imager 10 is capable of processing data and more particularly imaging data, motion and/or acceleration data, and the like. In some embodiments, the portable medical imager 10 is capable of communicating data wirelessly and/or via wired pathways.

The portable medical imager 10 includes a housing 12 configured to at least partially enclose any suitable number of components associated with the portable medical imager 10. For example, the housing 12 may enclose and support internally various electrical components (including integrated circuit chips and other circuitry) to provide computing operations for the portable medical imager 10. The integrated circuit chips and other circuitry include a microprocessor, memory, a battery, a circuit board, I/O, various input/output (I/O) support circuitry and the like. In one embodiment, the housing 12 is integrally formed in such a way as to constitute is a single complete unit. The housing 12 can be formed of any number of materials including for example plastics, metals, ceramics and the like.

Although the portable medical imager 10 may connect through various wired connections, it should be appreciated that this is not a limitation. In one embodiment, the electronic portable medical imager 10 also includes a mechanism for wireless communications. For example, as shown, the portable medical imager 10 may include an antenna. The antenna may be disposed internal to the housing 12. The wireless communications can be based on many different wireless protocols including for example Bluetooth, RF, 802.11, and the like.

The portable medical imager 10 also includes a display 14 configured to define/carry a user interface of the portable medical imager 10. The display, for example, provides a viewing region for a display screen 14 used to display the acquired image or a graphical user interface. In some embodiments, portable medical imager 10 provides a user interface (not shown) that can be used to provide a user input event to the portable medical imager 10. Such user input events can be used for any number of purposes, such as changing the image acquisition or reconstruction settings, selecting between display screens presented on display screen 14, and so on. In one embodiment, the viewing region of the display 14 is touch sensitive for receiving one or more touch inputs that help control various aspects of what is being displayed on the display screen.

In some embodiments, the portable medical imager 10 also includes one or more connectors for transferring data to and from the portable medical imager 10. The connector may be used to upload or down load image data, motion and/or acceleration data, physiological data, and the like as well as operating systems, applications and the like to and from the portable medical imager 10. For example, one or more remote medical devices that measure physiological parameters of the patient and generate physiological data indicative thereof may connected to the portable medical imager 10 utilizing the one or more connectors. These remote medical devices include ECG sensors, blood pressure sensors, pulse sensors, respiratory sensors, and the like. For example, the remote medical sensors are utilized to measure the heart and breathing motion present during the image acquisition. Of course, other remote medical devices can be associated with a patient, and not all of the above-mentioned remote medical devices have to be associated with the patient at any given time. It is also contemplated that the remote medical device communicate the physiological data via wireless communications based on many different wireless protocols including for example Bluetooth, RF, 802.11, and the like.

To acquire an image, the portable medical imager 10 is held over a patient (not shown) to be imaged. The patient includes a region of interest (ROI) to be imaged by the portable medical imager 10 Examples of the ROI include, but are not limited to, hearts, brains, thyroids, bones, joints, ligaments, tendons, muscles, nerves, kidneys, lungs, tumors, lesions, and so on. Before imaging, the ROI is injected with one or more radioisotopes including, but not limited to, Tc-99m, 1-131, Ga-67, In-111, and the like. In some embodiments, the radioisotopes are combined with radioligands to create a radiopharceutical that binds to or is preferentially absorbed by specific types of tissue.

The portable medical imager 10 includes one or more radiation detectors 16 for detecting radiation events and imaging the ROI, as discussed hereafter. Specifically, a frame 18 attached to the housing 12 of the portable medical imager 10 supports the one or more radiation detectors 16 of the portable medical imager 10. The detector, in one SPECT embodiment, includes a collimator, a scintillator layer, an array of light sensitive diodes, such as SiPMs. Solid state direct gamma radiation detectors are also contemplated. During imaging, the portable nuclear medicine image 10 is typically statically held by a user. No rotating gantry or moving patient support is required. Because the portable medical imager 10 typically remains static during imaging, the portable medical imager 10 includes a sufficient number of radiation detectors to capture the ROI for image reconstruction.

However, it is contemplated that the portable medical imager 10 can move during imaging. The portable medical imager 10 can move relative to the patient slightly during imaging. In order to account for the motion of the portable medical imager 10, one or more motion sensors 20 are utilized. The motion sensors 20 are configured to measure motion or acceleration, such as an accelerometer or a gyroscope. In one embodiment, the motion sensor 20 is a six-axis accelerometer that includes a sensing element and an integrated circuit interface for providing the measured acceleration and/or motion data to the portable medical imager 10. The motion sensor 20 may be configured to sense and measure various types of motion including, but not limited to, velocity, acceleration, rotation, and direction. The acceleration and/or motion data are utilized to compensate for motion of the portable medical imager 10 during the image acquisition as well as determine the position the radiation detectors 16 at different viewing angles of the ROI, as discussed hereafter. From the motion data, the location and orientation of the detector and/or a correction to trajectories of the incoming radiation are determined. In a portable nuclear image, the collimator controls the trajectory of the incoming radiation relative to the imager. As the detector moves, such as translates or rotates relative to a frame of reference, e.g. 3 orthogonal axes, the portable medical imager 10 uses the motion data to define a trajectory for each radiation event. It is also contemplated that one or more motion sensors 20 be utilized in assisting the position of one or more radiation detectors 16 relative to one another.

During imaging, the radiation detectors 16 receive gamma photons emitted by the radioisotopes injected into the ROI and generate radiation data indicating the location of the radioisotopes within the ROI. In some embodiments, the radiation detectors 16 are modular and share the same size and area (e.g., 32 mm×32 mm) as the portable medical imager 10. To secure the radiation detectors 16 to the frame 18, any approach of connecting the radiation detectors 16 is appropriate.

In some embodiments, the radiation detector 16 includes one or more scintillator elements generating light flashes when struck by gamma photons. The location of a light flash corresponds to the location of a gamma photon strike. In some embodiments, the scintillator elements are pixelated. Further, in some embodiments, the scintillation elements include a receiving face which receives the gamma photons emitted by the radioisotopes. When the receiving face receives a gamma photon, the scintillation elements emit a light flash at least partially from an output face (not shown) of the scintilliation elements, opposite the receiving face. Examples of scintillation elements include scintillator plates, individual scintillation crystals (e.g., sodium iodide crystals), and the like. In some embodiments, the scintillator elements are pixellated.

One or more light sensitive elements sense the light flashes generated by the scintillator elements and generate radiation data indicating the location on the imager and intensity of the light flashes. In some embodiments, the light sensitive elements are pixelated. For example, the pixels of the light sensitive elements can be larger than pixels of the scintillator elements and Anger logic can be employed for positioning the scintillation events. Further, in some embodiments, the light sensitive elements include a receiving face which receives the light flashes emitted by the scintillation elements. In such embodiments, the output face and the receiving face of the light sensitive elements are spatially correlated optically. The light sensitive elements then sense the location of light flashes by sensing the location of light flashes striking the receiving face of the light sensitive elements. Since the receiving face of the light sensitive elements are spatially correlated optically, locations on the receiving face of the light sensitive elements can be correlated with locations on the output face, which correspond to locations on the receiving face of the scintillator elements.

In one embodiment, the light sensitive elements include digital or analog silicon photomultipliers (SiPMs). While analog SiPMs are amenable to the present disclosure, suitably digital SiPMs are employed. Pixelated scintillator elements, in one embodiment, are coupled 1:1 with the SiPMs. It is also contemplated that the light sensitive elements can employ photomultiplier tubes, photodiodes, opto-electric transducers, direct photon to electrical converters (a.k.a., semiconductor gamma detectors), such as semiconductor crystals, zinc-cadmium telluride (CZT) elements, and the like, and so on.

Each radiation detector 16 further includes one or more collimators controlling the direction and angular spread from which each scintillator element of the radiation detector can receive radiation. In other words, the collimators ensure the scintillator elements receive radiation along known rays or trajectories. Typically, the collimators include one or more openings limiting the radiation received by the scintillator elements to the radiation that passes through the openings. Examples of collimators include pin-hole, slat-slit and fan beam-slit.

In a pin-hole or slat-slit collimator, the pin-hole or slat-slit functions analogous to a pin-hole camera to focus the radiation on the scintillator elements or solid state detector array. In the slat-slit camera, the slats limit the spread or divergence of the radiation trajectories in the direction of the slit.

In some embodiments, a plurality of the light sensitive elements, for example, six in a row, share a slat-slit or fan beam-slit collimator. Further, in some embodiments, the collimators are replaceable to allow the portable medical imager 10 to be employed for different imaging techniques. For example, collimators for SPECT imaging or planar imaging can be employed depending upon the desired imaging technique. In embodiments employing modular collimators, the collimators can be secured to the radiation detector using, for example, mechanical fasteners or a system of interlocking grooves.

It is also contemplated that the radiation detector 16 includes a single light sensitive element array, such as a digital SiPM array, and a single scintillator element, such as a plate of sodium iodide crystals.

A data acquisition processor 22 of the portable medical imager 10 collects radiation data from each of the radiation detectors 16 and the information from the motion sensor 20 for a predetermined period of time. For each of the radiation detectors 16, the radiation data typically includes energy of gamma photon strikes and the corresponding locations of the gamma photon strikes. In some embodiments, for each radiation detector, the data acquisition processor 22 uses the radiation data to generate a radiation distribution over the spatial range of the radiation detector.

The data acquisition processor 22 uses the received motion and/or acceleration data from the motion sensor 20 to determine the angular orientation and position of each radiation detector relative to the ROI and correlates translations and the angular orientations and positions with the received radiation data and/or radiation distributions. Using the detector location on the radiation detector and the motion data, the data acquisition processor 22 determines the trajectory (as well as the energy) of each received radiation event.

During imaging, the user moves the portable medical imager 10 to other locations around the patient to monitor the emitted radiation from a plurality of directions and angles. In this manner, radiation events are received along with a multiplicity of trajectories in three dimensions. The portable medical imager 10 collects radiation data from each of the radiation detectors 16 for a selected period of time at each of these locations. To properly correspond the image data to the various locations, the data acquisition processor 22 in one embodiment receives the motion and/or acceleration data from the motion sensor 20 and generates an angular position map for motion in x, y, and z-direction. This angular position map describes the angular position of the portable medical imager 10 for example relative to a ROI. The data acquisition processor 22 then correlates the angular positions with the received radiation data and/or radiation distributions based on the angular position map. The radiation data and/or the radiation distributions are stored in an image data memory 24 of the portable medical imager 10. Further, the angular positions of the radiations, correlated with the radiation data and/or radiation distributions, are further stored in the image data memory 24.

A reconstruction processor 26 of the portable medical imager 10 processes the data collected over the multiplicity of trajectories from the imaging data memory 24 into a three-dimensional image representation. In one embodiment, the data acquisition processor 22 defines the trajectories as discussed above. In another embodiment the image memory 24 stores the location on the detector and the time each event is received. The motion sensor 20 generates and saves the motion dta as a function of time. The reconstruction processor 26 then correlates the location and angularo orientation from the motion sensor 20 with the location on the detector to adjust for location and orientation changes during the image reconstruction using the time information.

In one embodiment, the motion and/or acceleration data includes the motion distance, direction, speed and the like is used to create a motion map which is used to correct the image data for motion along and rotation around x, y, and z-directions. In another embodiment, the reconstruction processor 26 also records time stamps with the measure locations of the event data which are used to correlate the location adjustments with the image data. It is also contemplated that the received physiological data is utilized to motion map relating the motion of the patient. For example, the physiological data received from the remote medical devices may be used to generate a motion map relating to the breathing cycle and/or heart rate of the patient. In some embodiments, the processing further includes generating a radiation distribution over the spatial range of each radiation detector from the radiation data. The image representation is stored in a reconstruction image memory 28 of the portable medical imager 10 for subsequent use. For example, the three-dimensional image can be employed by the portable medical imager 10 and/or displayed on a display 14. It is also contemplated that the generated image data is transmitted from the portable medical imager to a remote image processing system.

The 3D volume image in the memory 28 is processed to generate any of a variety of displays on the display 14. For example, one or more slice images are displayed, e.g. orthogal slice. A series of slices can be displayed in cine format. Volume rendering, maximum or minimum intensity images, hybrid images, and the like are also contemplated.

Although not described in detail, those skilled in the art will appreciate that the portable medical imager 10 can be modified for positron emission tomography (PET) imaging by removing the collimators and adjusting the reconstruction algorithm. Applications for such PET imaging include dosimetry control during proton therapy. The image can be used with a radiation source, also with a motion sensor, for transmission SPECT imaging. Further, the portable medical imager 10, whether employed for SPECT or PET imaging, can be combined with computed tomography (CT), volume imaging (XCT), or magnetic resonance (MR) for multimodal imaging by using just one patient support for both applications.

As used herein, a memory includes one or more of a non-transient computer readable medium; a magnetic disk or other magnetic storage medium; an optical disk or other optical storage medium; a random access memory (RAM), read-only memory (ROM), or other electronic memory device or chip or set of operatively interconnected chips; an Internet/Intranet server from which the stored instructions may be retrieved via the Internet/Intranet or a local area network; or so forth. Further, as used herein, a processor includes one or more of a microprocessor, a microcontroller, a graphic processing unit (GPU), an application-specific integrated circuit (ASIC), a field-programmable gate array (FPGA), and the like; and a user input device includes one or more of a mouse, a keyboard, a touch screen display, one or more buttons, one or more switches, one or more toggles, and the like.

With reference to FIG. 3, a method 100 for imaging the ROI is provided. In a step 102, a portable imaging device is positioned against a ROI of a patient. The portable imaging device including one or more radiation detectors disposed within the housing generating radiation data indicating the location of gamma photon strikes and a motion sensor which indicates a location and orientation of the detector at each strike. In a step 104, radiation data and motion data collected by at least one processor from the radiation detectors and the motion sensor. In a step 106, the portable device is repositioned to each of a plurality of other locations and/or orientations to receive radiation from the regions of interest over about 180 degrees. In a step 108, a 3D volume image of the ROI is reconstructed by the at least one processor from the received radiation data and motion data. In a step 110, a slice image or other depictions of all or part of the reconstructed image is displayed on a display of the portable imaging device.

The invention has been described with reference to the preferred embodiments. Modifications and alterations may occur to others upon reading and understanding the preceding detailed description. It is intended that the invention be constructed as including all such modifications and alterations insofar as they come within the scope of the appended claims or the equivalents thereof.

1. A portable imaging system for imaging a region of interest (ROI), said system comprising:
a housing;
a radiation detector mounted to the housing, the detetor generating radiation data indicating a location of gamma photon strikes on the detector;
a motion sensor which senses motion of the detector and outputs motion data indicative of the location and orientation of the detector at the time of each gamma photon strike;
at least one processor programmed to:
receive the radiation data from the radiation detectors and the motion data from the motion sensor; and
reconstruct an image of the ROI from the received radiation and motion data.

2. The system according to claim 1, wherein the portable nuclear imaging system is gantry free.

3. The system according to claim 1, wherein the portable nuclear imaging system includes at least one of a handheld or tablet PC.

4. The system according to claim 1, further including:
a display mounted to the housing to display reconstructed images of the ROI.

5. The system according to claim 4, wherein the display and the radiation detector are mounted parallel to each other on opposite faces of the housing.

6. The system according to claim 1, wherein the motion data is utilized to at least one of compensate for motion of the portable nuclear imaging system and determine an angular position radiation detectors at different viewing angles of the ROI as the system is manually positioned and held in position.

7. The system according to claim 1, further including:
a remote medical device which measures physioliogical parameters of the patient and generates physiological data indicative thereof.

8. The system according to claim 7, wherein the physiological data is utilized to compensate for motion of the patient.

9. The system according to claim 1, wherein the image of the ROI from the received radiation and motion data is a 3D volume image.

10. The system according to claim 1, wherein the radiation detector includes:
at least one scintillator element generating light flashes in response to gamma photon strikes; and
an array of light sensitive elements, such us digital silicon photomultipliers, generating the radiation data in response to the light flashes.

11. The system according to claim 1, wherein the radiation detector includes:
an array of solid state detectors which generate radiation data in response to gamma photon strikes.

12. A portable imaging system for imaging a region of interest (ROI), said system comprising:
a housing;
a radiation detector mounted to the housing to generate radiation data indicating the location of gamma photon strikes;
a display mounted to an opposite face of the housing;
a motion sensor mounted in or to the housing to sense movement of the housing and generate motion data indicative of the relative positions and orientations of the housing.

13. A portable imaging system for imaging a region of interest (ROI) according to claim 12, further including:
a reconstruction processor which receives the radiation data from the radiation detectors and the motion data from the motion sensor; and reconstructs an image of the ROI from the received radiation and motion data.

14. A method for imaging a region of interest (ROI), said method comprising:
positioning a portable imaging device facing a ROI of a patient, the portable imaging device including a detector disposed on the housing to generate radiation data indicating the location of gamma photon strikes and a motion sensor which senses motion of the detector and outputs motion data indicative of the location and orientation of the detector at the time of each gamma photon strike;
receiving with at least one processor the radiation data from the radiation detectors and the motion data from the motion sensor; and
reconstructing by the processor an image of the ROI from the received radiation and motion data.

15. The method according to claim 14, further including:
positioning the portable image device in each of a plurality of locations and orientations facing the ROI.

16. The method according to claim 4, further including:
displaying the reconstructed image on a display of the portable imaging device.

17. The method according to claims 16, further including:
determining an angular orientation and position of the radiation detector relative to the ROI and
correlating the angular orientation and position with the received radiation data.

18. The method according to claim 16, further including:
determining an angular position radiation detectors at different viewing angles of the ROI from the motion data.

19. The method according to claim 14, further including:
measuring physioliogical parameters of the patient and generating physiological data indicative thereof; and
compensating for motion of the patient with the physiological data.

20. The method according to claim 14, wherein the portable imaging, device manually positioned and held in position.
